(19) United States
(12) Patent Application Publication
(10) Pub. No.: US 2007/0073266 A1
(43) Pub. Date: Mar. 29, 2007

(54) Compact wireless biometric monitoring and real time processing system

(75) Inventors: Alan Chmiel, Avon Lake, OH (US); Bradley T. Humphreys, Lakewood, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: ZIN Technologies, Brook Park, OH (US)

(21) Appl. No.: 11/236,899

(22) Filed: Sep. 28, 2005

(51) Int. Cl. A61M 31/00; A61B 5/00

(52) U.S. Cl. 604/503; 604/66; 600/300

(57) ABSTRACT

Systems and methodologies that regulate in real time biometric indicia of an ambulatory patient via employing a distributed computing arrangement of modular component(s), which are tailored in part based on requirements of data to be measured and/or administered. Accordingly, the system can be scaled for different biometric requirements (e.g., data bits, operating frequencies and the like). Such an arrangement can regulate drug delivery units and/or acquire biometric data from an ambulatory patient.

### GOVERNMENT INTERESTS

This subject innovation developed with government support under Contract No. NNC05CA65C awarded by NASA. The United States government has certain rights in the innovation.

### BACKGROUND

Diagnosis of ailments and treatment of disease often requires an analysis of biological signs obtained from a patient in the course of normal activity over a period of time. Personal health monitors are commonly employed to gather data related to a patients biometric data.

In general, a personal health monitor is a device used to measure and record one or more clinical parameters of a patient for later transmission to the patient's physician or other health care provider. The personal health monitor may be used in a hospital or clinical setting as an adjunct to existing care. Additionally, the personal health monitor may also be used by the patient outside care facilities (e.g., at a patient's home). When used by a patient at home, the patient operates the personal health monitor to record certain bodily clinical parameters. The personal health monitor can be used by the patient who has a condition requiring monitoring of one or more clinical parameters, but who otherwise may not require the level of care such as provided by a hospital. Accordingly, the personal health monitor provides potential savings in medical costs involved with a hospital stay.

For example, continuously monitoring cardiac patients immediately following coronary attacks is important. Such is normally accomplished effectively in the coronary care unit of most hospitals where the patients are continuously monitored following heart attacks to detect arrhythmias of the heart, for example monitoring and warning for ventricular arrhythmias, which may lead to ventricular fibrillation and death. Through prompt recognition and treatment of such warnings related to ventricular arrhythmias in coronary care units, the mortality rate of acute myocardial infarctions has been reduced considerably. In addition, many post myocardial infarction cardiac patients continue have frequent ventricular extra systoles after discharge from the hospital. Accordingly, it is desired to continuously monitor the patient over a certain period of time and under varying conditions of stress, to determine the effectiveness treatment which has been introduced, such as the proper dosage of medication.

Constant monitoring of such patients after release from the hospital may be difficult because of the logistics involved, and particularly since they can no longer be monitored closely as a group by direct wiring or close telemetry, as commonly implemented in hospital settings. As a result, various systems have been developed to attempt to monitor the ECG signals of out-patients to thereby provide a diagnostic tool for additional treatment or variation of treatment for the patients as may be required. Accordingly, there has been a persistent need to develop health monitoring systems and methods that can effectively alert medical personnel when a patient needs medical assistance.

Nevertheless, such mobile units are typically spacious and difficult to set up and maintain. Moreover, in general these units are not suitable for readily monitoring a plurality of biological signs and biometric indicia. In addition, such systems lack flexibility during usage as they typically have fixed sensor types and configurations.

At the same time, compatibility of such systems with various communication requirement and protocols can create further problems and increase costs. This can further hinder a quick response of the medical staff when health issues arise for an ambulatory patient who employs such monitors. Also, with the current limits in resolution on existing biometric data acquisition modules, the analysis of low magnitude (and sometimes long duration) of various biometric parameters (e.g., EKG activity) is typically hindered and/or not possible. Such problem is further compounded due to gain amplifiers lack of operation flexibility, wherein the gain amplifiers (e.g., associated with sensors) are commonly set for high exertion activity levels.

Therefore, there is a need to overcome the aforementioned exemplary deficiencies associated with conventional systems and devices.

### SUMMARY

The following presents a simplified summary of the innovation in order to provide a basic understanding of one or more aspects of the innovation. This summary is not an extensive overview of the innovation. It is intended to neither identify key or critical elements of the innovation, nor to delineate the scope of the subject innovation. Rather, the sole purpose of this summary is to present some concepts of the innovation in a simplified form as a prelude to the more detailed description that is presented hereinafter.

The subject innovation provides for systems and methods of regulating in real time biometric parameters/indicia of an ambulatory patient via employing a distributed computing arrangement of modular component(s), which are tailored in part based on requirements of data to be measured and/or drugs administered. The modular component can include a plurality of cards grouped together (e.g., flash cards, memory cards, smart cards, flash memory devices, communication card, data acquisition circuitry and the like) as part of a package with an interconnect to a sensor. By replacing, inserting, swapping a card, the modular component can be tailored to operate for acquisition of a particular biometric data and/or transmit data based on a particular transmission protocol.

For example, the modular component can be tailored to acquire data related to Electromyography (EMG, frequency range 2-500 Hz), Electrocardiography (ECG, frequency range 0.05-100 Hz, resolution of 24 bits), Electroencephalography (EEG, frequency range 0.16-100 Hz), blood pressure, and the like. Accordingly, the system can be scaled for different biometric requirements (e.g., data bits, operating frequencies and the like). Such an arrangement of modular components can further adapt to a plurality of communication protocols by supplying associated communication card, and transceive data related to the biometric indicia to remote units (e.g., laptops, personal digital assistants, computing units, servers, and the like).

In a related aspect, a master processor as part of a master controller of the system can be operatively connected to at least one slave processor, wherein each slave processor is associated with a respective modular component, for example. As such, the slave processor on each modular component can obtain data at a predetermined rate (e.g., a programmable rate) based on type of data which the modular component is to acquire. Data can be acquired asynchronously, wherein different modular components with different sensor requirements can acquire data at different sample rates. The subject innovation enables, asynchronous data collection across modules, while at the same time supplying a synchronous clock to provide timing on module for data collection functions. Moreover, auto-ranging can be provided for gain settings of amplifiers associated with the modular component to avoid a saturation of the amplifiers, (e.g., for EMG variations of a sedentary patient, and also during exercise).

According to a further aspect of the subject innovation, the master processor can be part of a master controller that controls high level functions of the system such as: Bus Traffic control, External data transmission, User Interfaces, System status Monitoring, Internal Data Storage and Retrieval, and the like. In a further aspect of the subject innovation, artificial intelligence components can also be employed for biometrics data acquisition/drug delivery administration.

To the accomplishment of the foregoing and related ends, the innovation, then, comprises the features hereinafter fully described. The following description and the annexed drawings set forth in detail certain illustrative aspects of the innovation. However, these aspects are indicative of but a few of the various ways in which the principles of the innovation may be employed. Other aspects, advantages and novel features of the innovation will become apparent from the following detailed description of the innovation when considered in conjunction with the drawings.

### BRIEF DESCRIPTION OF THE DRAWINGS

FIG. 1 illustrates a block diagram of a distributed computing environment in accordance with an aspect of the subject innovation.

FIG. 2 illustrates a perspective diagram of modular component that includes a plurality of cards packaged together.

FIG. 3 illustrates a block diagram of an exemplary modular component that can acquire biometric data for real time monitoring and drug delivery.

FIG. 4 illustrates a spatial distribution of modular components around a patient.

FIG. 5 illustrates a perspective for packaging of a modular component, wherein cards can be replaced, inserted or swapped for desired operation.

FIG. 6 illustrates a schematic diagram of the modular component of the subject innovation that interacts with a plurality of clients and/or remote units.

FIG. 7 illustrates a particular ECG measurement block diagram in accordance with an aspect of the subject innovation.

FIG. 8 illustrates a particular EMG measurement block diagram in accordance with an aspect of the subject innovation.

FIG. 9 illustrates a particular Electroencephalogram (EEG) measurement block diagram in accordance with an aspect of the subject innovation.

FIG. 10 illustrates a particular block diagram for a Pulse Oximeter block diagram in accordance with an aspect of the subject innovation.

FIG. 11 illustrates a Joint angle measurement block diagram for detecting range of motion for joints.

FIG. 12 illustrates a block diagram associated with a modular component that measures pressures on the sole of a patient's foot (Plantar Pressure).

FIG. 13 illustrates a methodology of acquiring biometric parameters.

FIG. 14 illustrates a further methodology of biometric data acquisition/transmission.

FIG. 15 illustrates an exemplary environment for implementing various aspects of the subject innovation.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the subject innovation. It may be evident, however, that the subject innovation may be practiced without these specific details. In other instances, well-known structures and devices are shown in block diagram form in order to facilitate describing the subject innovation.

As used herein, the terms “component,” “system” and the like, in addition to electro-mechanical components, can also refer to a computer-related entity, either hardware, a combination of hardware and software, software, or software in execution.

For example, a component may be, but is not limited to being, a process running on a processor, a processor, an object, an executable, a thread of execution, a program, and/or a computer. By way of illustration, both an application running on computer and the computer can be a component. One or more components may reside within a process and/or thread of execution and a component may be localized on one computer and/or distributed between two or more computers. Also, the word “exemplary” is used herein to mean serving as an example, instance, or illustration. Any aspect or design described herein as “exemplary” is not necessarily to be construed as preferred or advantageous over other aspects or designs.

Additionally it should be appreciated that a carrier wave can be employed to carry computer-readable electronic data such as those used in transmitting and receiving electronic mail or in accessing a network such as the Internet or a local area network (LAN). Of course, those skilled in the art will recognize many modifications can be made to this configuration without departing from the scope or spirit of the claimed subject matter.

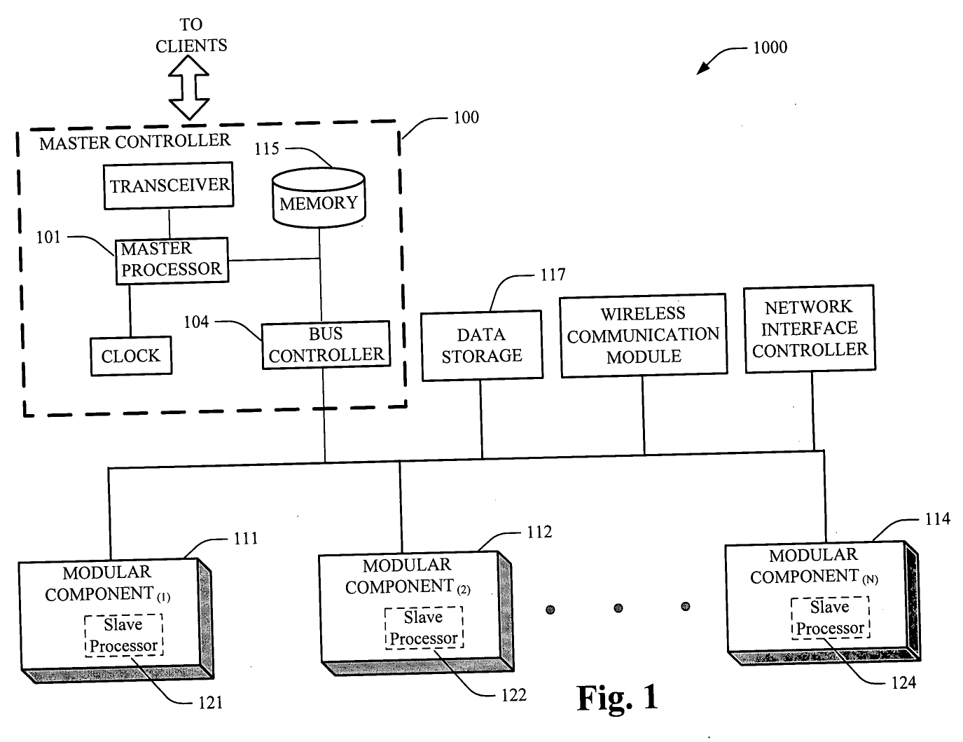

FIG. 1 illustrates a block diagram of a system 1000 that regulates in real time biometric indicia of a patient via employing a distributed computing arrangement of modular components 1 thru N (where N is an integer) 111-114. Such modular components 111-114 are tailored in part based on requirements of biometric data to be measured and/or administered. Accordingly, the system can be scaled for different biometric requirements (e.g., data bits, operating frequencies and the like). Each of he modular components 111-114 can include a plurality of cards grouped together (e.g., flash cards, memory cards, smart cards, flash memory devices, communication card, modality specific modules such as specific data acquisition circuitry and the like) as part of a package with an interconnect to a sensor. By replacing, inserting, swapping a card, the modular components 111-114 can be tailored to operate for acquisition of a particular biometric data and/or transmit data based on a particular transmission protocol. Such an arrangement of modular components 111-114 can further transceive data associated with the biometric indicia to remote units (e.g., laptops, personal digital assistants, computing units, servers, and the like), as described in detail infra. The system 1000 includes a master control processing unit CPU 101 as part of a master controller 100. The CPU 101 can control high level functions including Bus Traffic control, External data transmission, User Interfaces, System status Monitoring, Internal Data Storage and Retrieval, and the like. A swappable communication card can configure communication between the modular component and the control system, to a predetermined protocol.

The modular components 111-114 can acquire biometric parameters associated with a patient, wherein modality specific modules (e.g., specific sensor circuitry for EKG, ECG, and the like) can be replaced, inserted and/or swapped for collection of biometric parameters. A clinician can then readily designate a routine and determine which modality specific modules and/or circuitry should be inserted into which modular component 111-114. Moreover, according to a control program or routine supplied by the CPU 101, a modular component can measure one or more biometric parameters, and/or supply input that is representative of the status of a controlled process, to compatible drug delivery units for example, and change outputs effecting control of the process. For example, the modular component 111-114 can supply activation commands to a glucose pump in a patient's proximity, when acquired data that pertains to blood sugar of a patient indicates a critical level. Similarly, muscle tension can be employed as a biometric indicia to be collected by a modular component, and employed for delivery muscle relaxation drugs by the same or other modular component to a patient. The inputs and outputs of the modular component can be binary, (e.g., on or off), and/or analog assuming a continuous range of values.

The control routine (e.g., supplied by the CPU 101) may be executed in a series of execution cycles with batch processing capabilities, and can interact with one or more functional units operably connected to the modular components 111-114, such as a glucose pump, and the like for drug delivery. Likewise, the measured inputs received from a the modular components 111-114 and/or controlled process and the outputs transmitted to the process may pass through one or more input/output (I/O) modules associated with the control system 1000, and can serve as an electrical interface between the modular components 111-114 and the controlled process, for example. Moreover, the inputs and outputs may be recorded in an I/O table in processor memory 115, 117. Input values such as a patient's biometric data (e.g., temperature, blood sugar level, and the like) can be asynchronously read via sensor of the modular component and output values can be written directly to the I/O table by slave processors 121-124 for subsequent communication to the process by specialized communications circuitry.

During execution of a control routine, (e.g., real time monitoring of blood sugar level), values of the inputs and outputs exchanged with and/or acquired by the modular components 111-114 and/or controlled process can pass through the I/O table. The values of inputs in the I/O table may be asynchronously updated from the controlled process by dedicated modular components. Moreover, modality specific circuitry can communicate with input and/or output modules over a bus on a backplane or network communications. The modality specific circuitry can also asynchronously write values of the outputs in the I/O table to the controlled process. The output values from the I/O table can then be communicated to one or more of the modular components 111-114 and/or associated output modules for interfacing with the process. Thus, a slave processor(s) 121-124 can simply access the I/O table rather than needing to communicate directly with the master processor and/or controlled process.

For example, the modular component(s) 111-114. can be operatively connected to a drug delivery system with an actuating mechanism, a delivery tube and a handle terminating with a needle, for example. Moreover, a syringe (or other fluid storage device) can be mounted upon the actuating mechanism with one end of tube being coupled to the syringe. The actuating mechanism can operate a plunger to selectively eject fluid out through the tube handle, and needle or alternatively to draw fluid in. The actuating mechanism can be controlled via the modular component thru selected values from the I/O table and/or various operational parameters discussed herein.

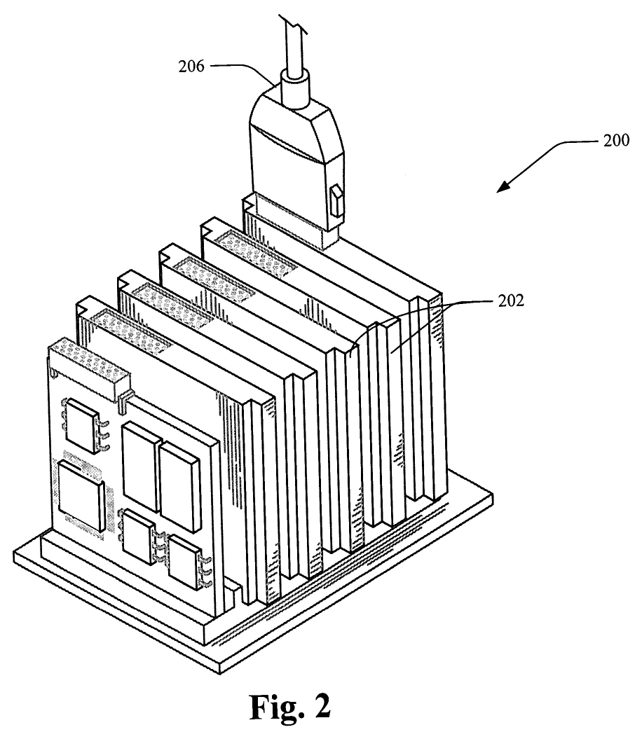

FIG. 2 illustrates a perspective view of a modular component 200 in accordance with an aspect of the subject innovation. Such modular component 200 includes a plurality of cards grouped together 202 (e.g., flash cards, memory cards, communication card, data acquisition circuitry and the like) as part of a package with an interconnect 206 to a sensor. By replacing, inserting, swapping a card, the modular component 200 can be readily tailored to operate for acquisition of a particular biometric data and/or transmit data based on a particular transmission protocol. For example, the modular component 200 can be adapted to acquire data related to Electromyography (EMG, frequency range 2-500 Hz), Electrocardiography (ECG, frequency range 0.05-100 Hz, resolution of 24 bits), Electroencephalography (EEG, frequency range 0.16-100 Hz), blood pressure, and the like.

As such, the slave processor on each modular component can acquire data at a rate required for data which the modular component is to acquire. Data can be acquired asynchronously, wherein different modular components with different sensor requirements can acquire data at different sample rates. Such enables, asynchronous data collection across modules, while at the same time employing a synchronous clock to provide timing on module for data collection functions.

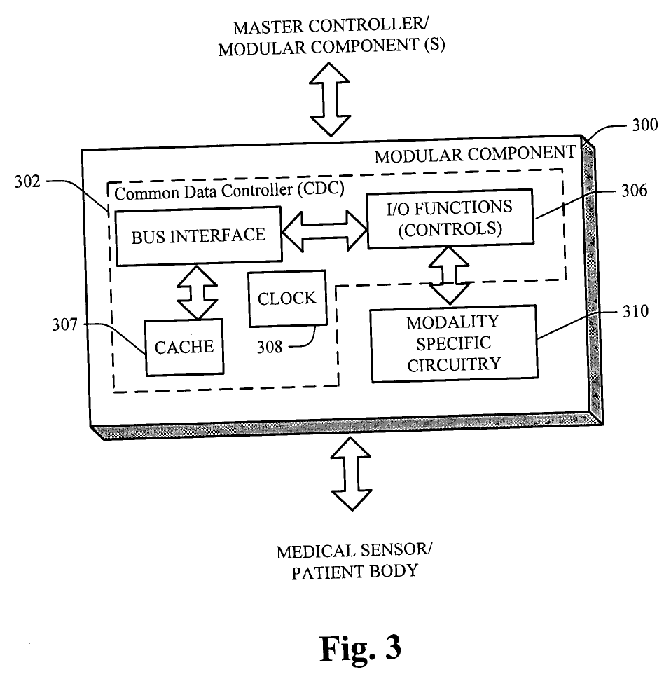

FIG. 3 illustrates a block diagram of modular component 300 that acquires biometric parameters and/or regulates such biometric indicia. The modular component 300 can include a Common Data Controller 302, which has a Bus Interface 302, I/O functions (controls) 306, and a module clock 308. The Bus Interface 302 can coordinate activities of the modular component 300 with a bus controller of the master controller (not shown), for transmittal of biometric indicia (e.g., medical parameter data) and reception of control data.

Likewise, the I/O functions 306 can control operation for the modality specific circuitry 310 (e.g., specific to EKG, EEG, and the like). Typically, the modular component 300 (e.g., required for a control task, such as monitoring blood sugar and control thereof in real time) can be connected to other modular components on a common backplane through a network or other communications medium. As explained earlier, the modular component 300 can include processors, power supplies, network communication modules, and I/O modules exchanging input and output signals directly with the master controller and/or the controlled process. Data may be exchanged between modules using a backplane communications bus, which may be serial or parallel, or via a network.

In addition to performing I/O operations based solely on network communications, smart modules can be employed that can execute autonomous logical or other control programs or routines. A RAM memory medium 307 can function as a data storage medium for buffering of collected, so that data is not lost when the system bus is in use by other functions. Such memory 307 also enables asynchronous data collection. Additionally, the module clock 308 provides for timing on a modular component for data collection functions. The module clock 308 supplies timing for data collection functions, and enables synchronous collection of data for the modular component 300, and asynchronous functions across modular components.

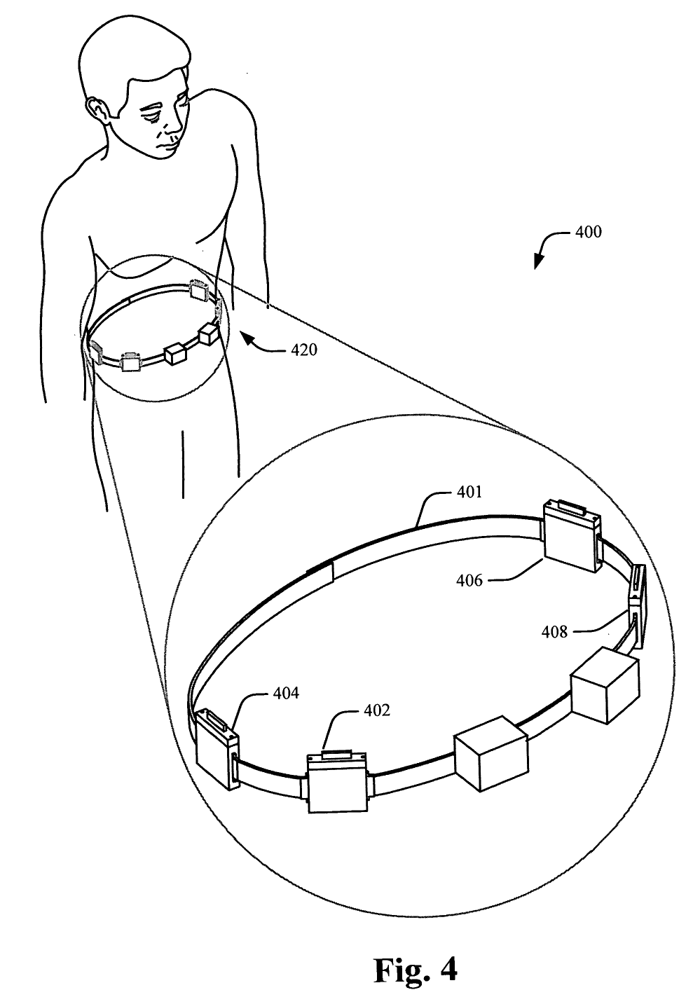

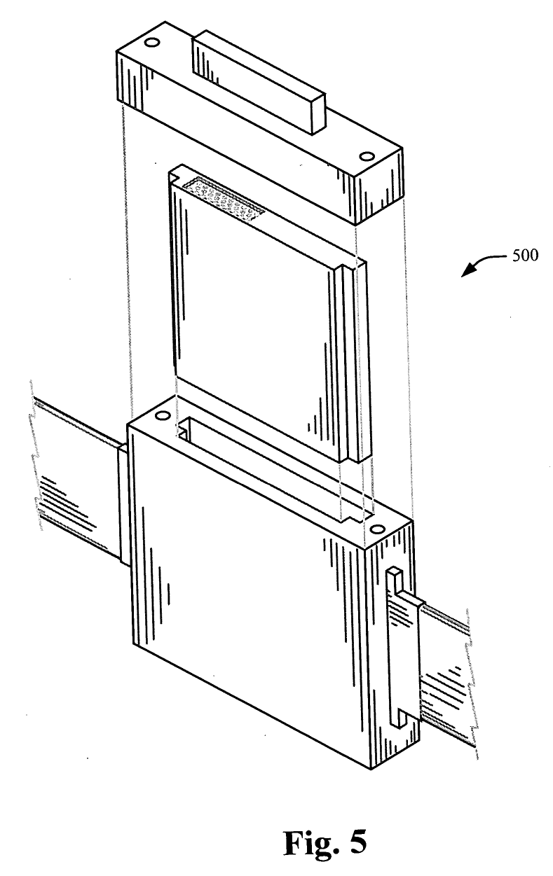

It is to be appreciated that various modular components for a distributed control system 400 may be spatially distributed along a common communication link, such as a belt 401 around a user's body as illustrated in FIG. 4. Certain modular components 402-408 can thus be located proximate to predetermined portions of a patient's body 420. Data can be communicated with such modular components 402-408 over a common communication link, or network, wherein all modules on the network communicate via a standard communications protocol. Like wise FIG. 5 illustrates a broken perspective for packaging of a modular component 500, wherein cards can be replaced, inserted or swapped for desired operation.

In such a distributed control system, one or more I/O modules are provided for interfacing with a process, wherein the outputs derive their control or output values in the form of a message from a master controller over a network or a backplane. For example, a modular component can receive an output value from a processor, via a communications network or a backplane communications bus. The desired output value for controlling a device associated with biometric indicia can be generally sent to the output module in a message, such as an I/O message. The modular component that receives such a message can provide a corresponding output (analog or digital) to the controlled process. The modular component can also measure a value of a process variable and report the input values to a master controller or peer modular component over a network or backplane. The input values may be used by the master processor for performing control computations.

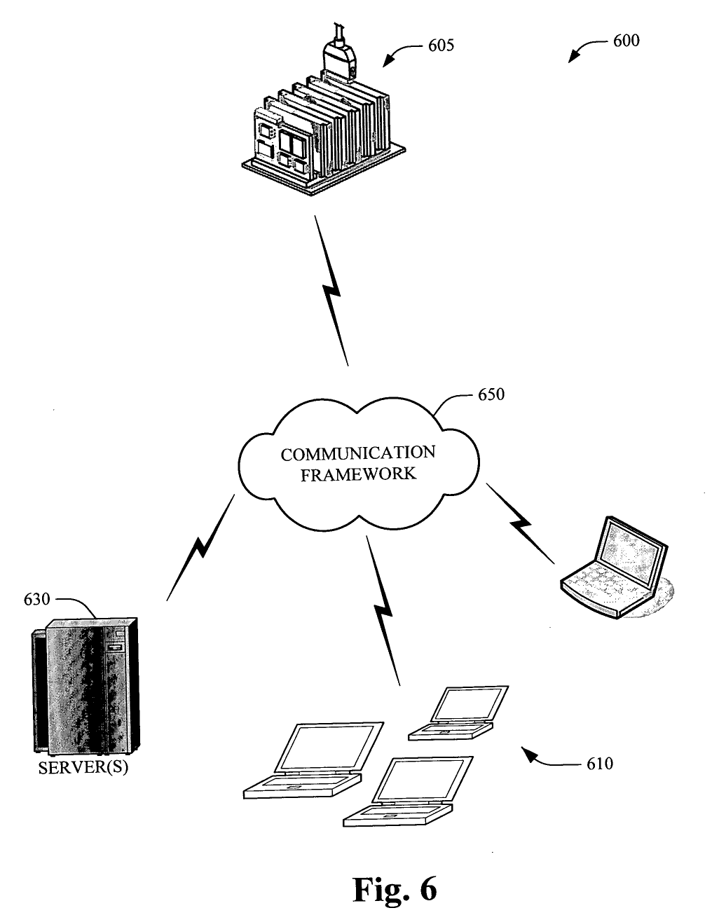

FIG. 6 illustrates a schematic diagram of the modular component 605 of the subject innovation that interacts with a plurality clients 610 and/or remote units. Data can be acquired through a compact (e.g., cell phone sized) modular components attachable to a patient, wherein data is then transmitted wirelessly to clients 610 such as PDA (Personal Digital Assistant), computing units, servers and the like, and viewed in real time by a clinician. The client(s) 610 can be hardware and/or software (e.g., threads, processes, computing devices). The system 600 also includes one or more server(s) 630. The server(s) 630 can also be hardware and/or software (e.g., threads, processes, computing devices). The servers 630 can house threads to perform transformations by employing the components described herein, for example. One possible communication between a modular component 605 a, client 610, and a server 630 may be in the form of a data packet adapted to be transmitted between two or more computer processes. The system 600 includes a communication framework 650 that can be employed to facilitate communications between the modular component 605, the client(s) 610 and the server(s) 630. The client(s) 610 can be operably connected to one or more client data store(s) that can be employed to store information local to the client(s) 610. Similarly, the server(s) 630 can be operably connected to one or more server data store(s) that can be employed to store information local to the servers 630. When out of range of the modular component, data can be stored onboard the monitoring device for later transmission. Such an arrangement can enable real time data streaming to clients extending the dynamic range of biometric signals that can be recorded, increase on-board memory capacity of the modular components, add auto-ranging gains for associated amplifiers, and provide additional instantaneous feed back to users through an extended local processing.

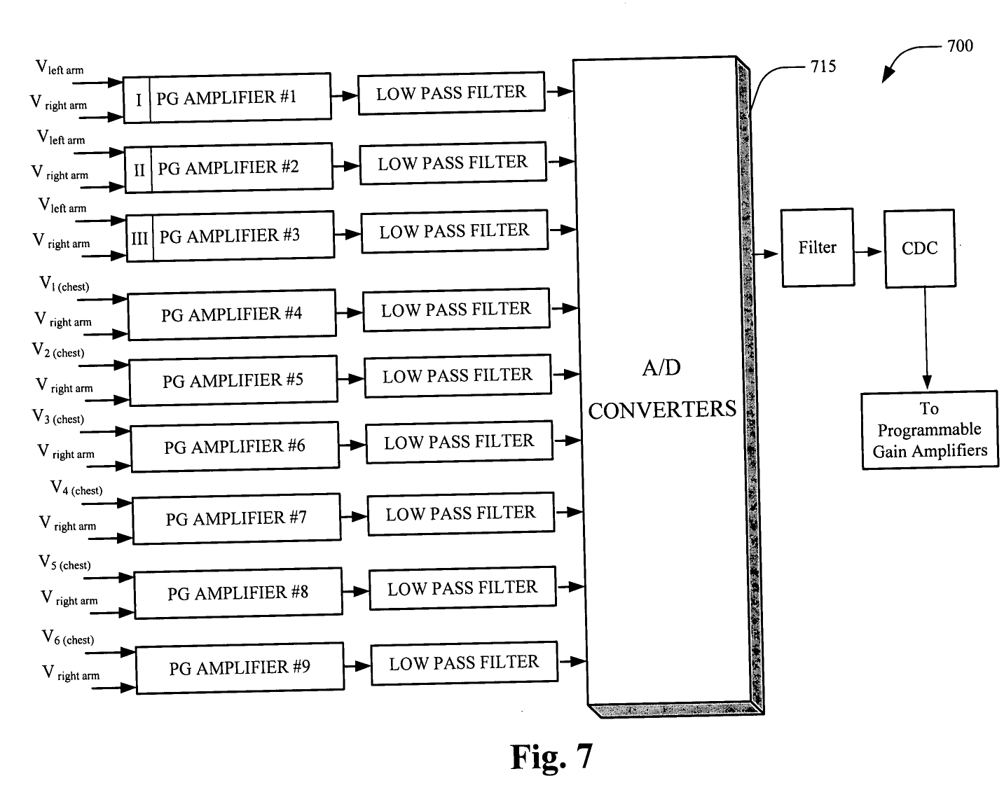

FIG. 7 illustrates a particular ECG measurement block diagram 700 in accordance with an aspect of the subject innovation. As explained earlier, the modular component can include a plurality of cards and/or be built from a set of configurable modules. Such modules can be configured for the unique needs of the subject or study. For example, the monitoring unit can record up to 80 channels of data from a variety of different sensors. These sensors include, but are not limited to Electromyography (EMG), Electrocardiography (ECG), Electroencephalography (EEG), Plantar Pressure, Joint Angle, Pulse Oximeter, Blood Pressure, Core Temperature, Blood Glucose, and the like. Each channel of data has independent programmable gain and isolation amplifiers. Each analog signal can then be recorded by a 24-bit Sigma Delta (ΣΔ) analog to digital converter 715. In addition, each channel can be individually configurable from 10 Hz to 1000 Hz sample rate, with a total maximum data throughput exceeding 32 kHz. Each channel has a minimum of 120 dB dynamic resolution and has an individual set of programmable filters to allow for real-time data filtering.

The monitoring unit's resolution can enable acquisition of low level parameters that over extended periods impact long term patient's health. For example, EMG data during periods of relatively low muscle exertion activity will be acquired and be discernable. An auto ranging feature associated with gain amplifiers for sensors of the subject innovation can facilitate resolution enhancement for biometric data acquisition. Typically, the electrocardiogram (ECG) and Electromyogram (EMG) module accommodates capture and digitization of analog data from both ECG and EMG sensors. ECG and MG sensors measure voltage differential across the surface of the patient's body. The ECG/EMG Module can have 16 differential inputs. 16 available inputs support the typical 3, 6, or 12 lead ECG measurement. In addition, ECG frequencies of interest are typically less than 500 Hz. For example, three and six lead ECG utilize three electrodes; twelve lead ECG employ 10 electrodes. Additionally, twelve “leads” can be calculated by taking the differential across specific pairs of electrodes. It is to be appreciated that the above exemplary implementation does not show the “right leg driver” terminal, and such terminal can be used to drive some small current, normally in the micro-amps, into the patient.

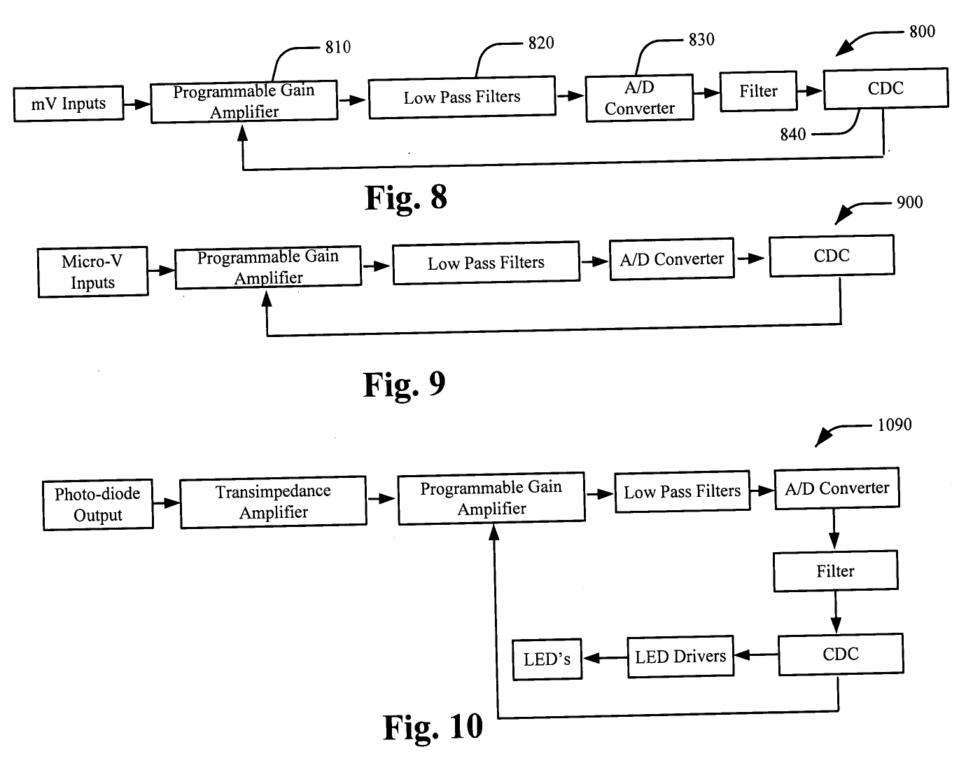

FIG. 8 illustrates a particular EMG measurement block diagram 800 in accordance with an aspect of the subject innovation. Typically, for EMG frequencies of interest are less than 500 Hz. The analog signal conditioning starts with a fully differential programmable gain amplifier (PGA) 810. In general, the principle function of the PGA is to calculate the differential potential between two passive single ended sensors. In addition, analog amplification of the signal can be performed, if desired. The fully differential PGA used in the subject innovation can generate very low distortions at higher gains. For example, the PGA 810 can improve the effective resolution by as much as 24 dB. The gain of the PGA 810 can be programmed by the processor through the Common Data Controller (CDC) 840. In an exemplary aspect, the Common Mode Rejection Ratio (CMRR) of the differential amplifier can be 125 dB. In general, CMRR is a measure of the ability of the differential input circuit to reject interfering signals that are common to both the input leads. The input impedance of the PGA 810, and hence the sensor interface of the module can be greater than 1 GΩ. Such high input impedance can facilitate reduction of the time constant of the system; which can significantly reduce the noise floor of the system. Moreover, the high input impedance further complies with FDA and related standards for medical device patient leakage current requirements.

After the PGA 810, the signal passes through a second order active low pass filter 820. Typically, an analog filter can act as an effective tool for reducing noise before digitization. In this exemplary implementation, the analog filter 820 is designed to allow the fundamental signals of interest to pass and maximize the rejection of out of band noise. The analog filter's frequency response is desired to fall to the stopband before reaching ½ of the next harmonic.

An analog filter can be designed to reduce the noise and provide a cleaner signal to the ADC. In one aspect, the analog filters in the ECG/EMG Module are designed to effectively eliminate out of band noise for the largest passband frequency. Such can increase overall system resolution by removing out of band noise before digitization, and facilitate reduction of quantized noise that is spread out over the spectrum by an associated modulator. The system 800 then relies on the implementation of the digital filters to supply high-resolution data. For example, the passband of the analog filter in this module can be 1000 Hz.

After the low pass filter 820, the signal is digitized by a high order, 24 bit, Sigma-Delta (ΣΔ) Modulator 830. A ΣΔ modulator can be designed to oversample the incoming data stream; and the output is then decimated. Such exemplary type of conversion can reduce the analog filtering requirements and the noise is spread out over a wider bandwidth. In addition, such an approach can be advantageous for lower bandwidth signals that require low noise, high-resolution digitization.

In general, the choice of modulator has a dramatic impact on overall system resolution. For example, not only does the number of bits help to achieve the overall system resolution, but also the order of the modulator and the effective oversampling ratio also affect the overall system noise. Equation 1 shows the effect that modulator order and oversampling ratio have on system noise.

$${n}_{0} = {e}_{RMS} ( \frac{{}^{M}}{\sqrt{2 M + 1}} ) {( 2 \frac{{f}_{o}}{{f}_{s}} )}^{M + \frac{1}{2}} \qquad [ Eq . 1 ]$$

wherein, e_{RMS} is the modulation noise of the converter, M is the number of loops (an integer) or order of the modulator and ƒₒ/ƒₛ, is the oversampling ratio.

FIG. 9 illustrates a particular Electroencephalogram (EEG) measurement block 900 block diagram in accordance with an aspect of the subject innovation. Electroencephalogram (EEG) is employed to measure electrical potentials produced by the brain. EEG measurement does not typically have the rigidity of measurement technique as ECG. For example, in classical techniques the placement of the common electrode and calculation of the differential pairs can be application specific. The number of leads can also be application specific, wherein the number of leads may be as high as 19 for the classical system, and as low as three for some clinical tests. Typically, for research applications, as many as 64 electrodes may be desired. Moreover, EEG signal levels are on the order of microvolts (μV). The frequency range of interest for EEG does not typically exceed 100 Hz. As the module is in general only capable of 16 differential measurements, modules can be used in parallel. If all five slots are populated with EEG modules a total of 80 channels can be recorded and correlated. The flow of the signal through the EEG module is substantially identical to that of the ECG module.

Likewise, FIG. 10 illustrates a particular block diagram 1090 for a Pulse Oximeter Module in accordance with an aspect of the subject innovation. The Pulse Oximeter can be employed to measure several parameters, including heart rate and the percent of arterial oxygen saturation (SaO₂). Such can require that red, or near IR light emitting diodes (LED's) be employed and the wavelength of light returned measured. Such measurement is typically taken at 60 Hz. The return signal is measured with a photo-transistor which has an output in the micro-to-milli amperes range. To accommodate this measurement, the module must typically first drive the LED'S at a fixed voltage. Such accomplished with a pulse width modulated voltage control circuit. Such circuit, similar to the entire module, is controlled through the CDC. The current returned from the prototransistor needs to be converted to voltage for digitization.

In one exemplary aspect a catch resistor can be employed and the V=IR relationship used to convert the current to voltage, in conjunction with a transimpedance amplifier with a gain of one. (The capacitance of the sensor works with the resistor to create a large time constant and can significantly raise the noise floor of the system. To avoid this, a transimpedance amplifier with a gain of one is used.)

The transimpedance amplifier can improve the response time by a factor of five or ten over a catch resistor. Moreover, the transimpedance amplifier also allows for more efficient control of the noise floor amplification. From the transimpedance amplifier the voltage is sent to the PGA. Subsequently, the signal follows the same flow as previously discussed in detail infra.

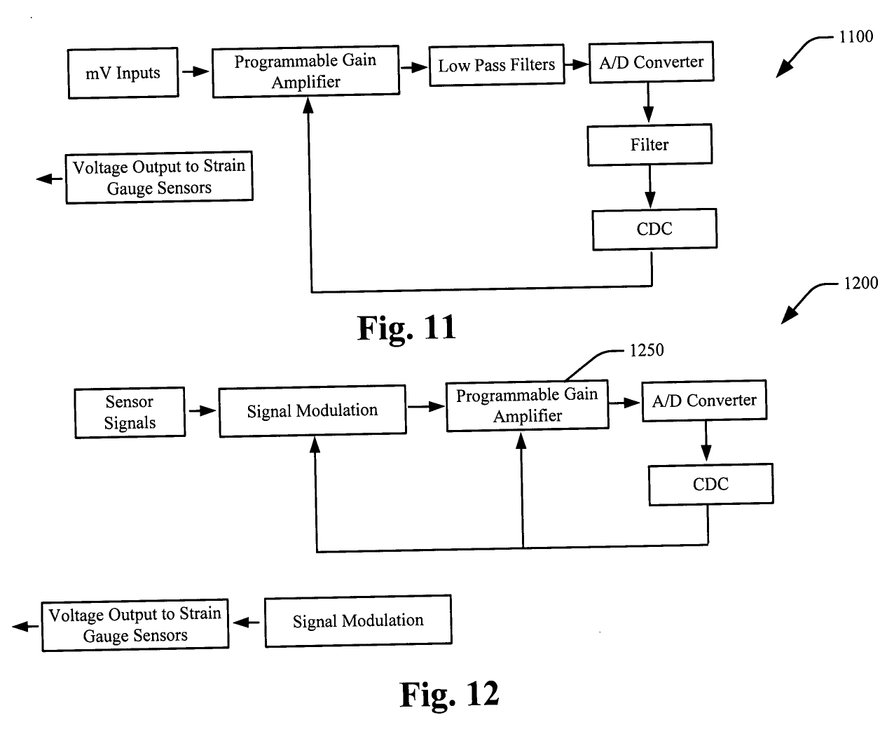

Referring now to FIG. 11, there is illustrated a Joint angle measurement block diagram 1100 for detecting range of motion of joints. The strain gauge measurement can be accomplished using a Wheatstone bridge configuration. In general, a Wheatstone bridge is a network of four resistances. It is used to measure an unknown electrical resistance by balancing two legs of a bridge circuit, one leg of which includes the unknown component. When voltage is applied across the bridge differential potential is measured between the legs. Frequencies of interest for this measure can be up to 128Hz. The Joint Angle Module has a flexible design allowing for measurement of a single or dual active leg. The differential voltage across the electro-goniometers is collected and passed through the programmable gain amplifier. Subsequently, the signal follows the same flow as discussed in detail infra.

Similarly, FIG. 12 illustrates a block diagram 1200 associated with a modular component that measures pressures on the sole of a patient's foot (Plantar Pressure). Such measurement is typically taken at frequencies less than 128 Hz. The active range is 20-600 kPa, where the measurement of pressure is directly taken as a capacitance measurement in the insole. The measurement can be taken by applying a modulated voltage across a capacitive bridge network. The change in voltage between the legs is related to the pressure on the insole. The differential voltage returned can be demodulated through a full wave rectifier and low pass filter and sent to the PGA 1250. Subsequently, the signal follows the same flow as discussed in detail infra.

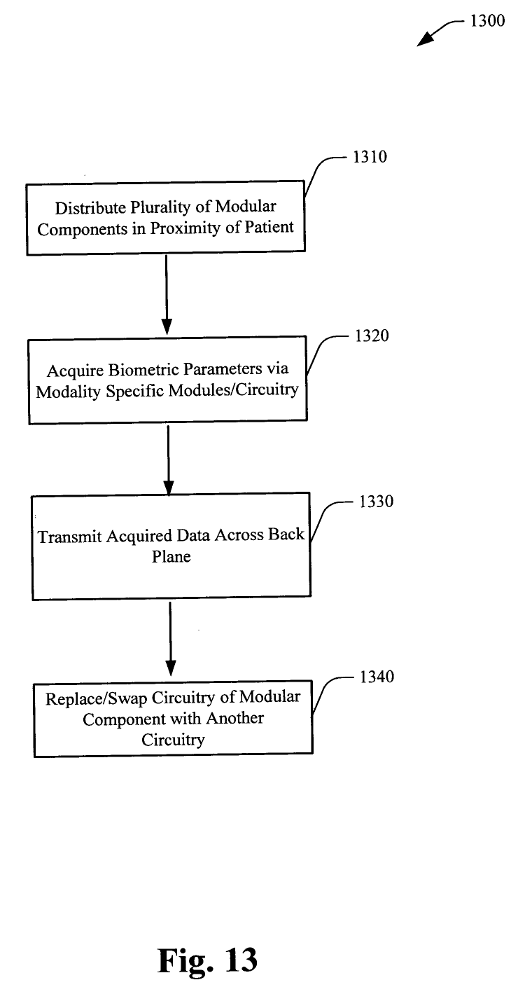

FIG. 13 illustrates a related methodology 1300 in accordance with an exemplary aspect of the subject innovation. While the exemplary method is illustrated and described herein as a series of blocks representative of various events and/or acts, the subject innovation is not limited by the illustrated ordering of such blocks. For instance, some acts or events may occur in different orders and/or concurrently with other acts or events, apart from the ordering illustrated herein, in accordance with the innovation. In addition, not all illustrated blocks, events or acts, may be required to implement a methodology in accordance with the subject innovation. Moreover, it will be appreciated that the exemplary method and other methods according to the innovation may be implemented in association with the method illustrated and described herein, as well as in association with other systems and apparatus not illustrated or described. Initially and at 1310, a plurality of modular components can be distributed in proximity to a patient. Such modular components include a plurality of cards grouped together (e.g., flash cards, memory cards, communication card, data acquisition circuitry and the like) as part of a package with an interconnect to a sensor. At 1320 biometric parameters can be acquired via modality specific modules/circuitry (e.g., sensors for EKG, ECG, and the like). Acquired data can then be transmitted across a back plane, to be monitored in real time by clinicians, at 1330. A segment of the modular component can then be replaced, or swapped with another module/circuitry to collect additional biometric data and/or tailor the device to a particular communication protocol.

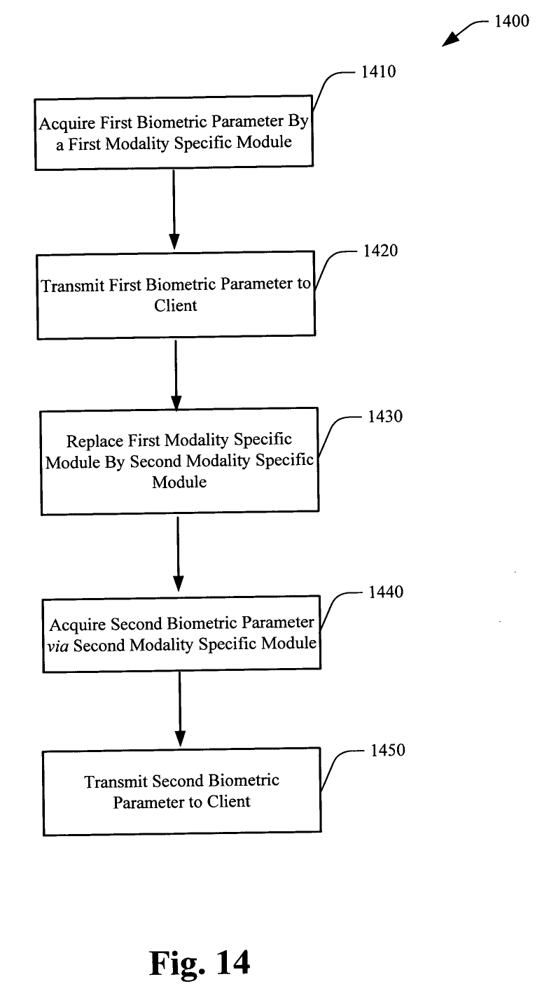

Referring now to FIG. 14 a related methodology 1400 of biometric data acquisition is illustrated. Initially, and at 1410 a first biometric parameter is acquired by a first modality specific module. Subsequently and at 1420 such first biometric parameter is wirelessly transmitted to clients of the system (e.g., physicians laptops, PDAs and the like). At 1430 the first modality specific module is replaced by a second modality specific module. Next, and at 1440 a second biometric parameter can be acquired via the second modality specific module and transmitted to the clients at 1450.

As such modality specific modules (e.g., for EKG, ECG, and the like) can be replaced, inserted and/or swapped for collection of biometric parameters. Thus, a clinician can tailor the system and determine which modality specific modules should be inserted into which modular component.

The subject innovation (e.g., in conjunction with regulating drug delivery and/or biometric data acquisition) can employ various artificial intelligence based schemes for carrying out various aspects thereof. For example, a process for learning explicitly or implicitly when and to what extent a drug should be employed can be facilitated via an automatic classification system and process. Classification can employ a probabilistic and/or statistical-based analysis (e.g., factoring into the analysis utilities and costs) to prognose or infer an action that a user desires to be automatically performed. For example, a support vector machine (SVM) classifier can be employed. Other classification approaches include Bayesian networks, decision trees, and probabilistic classification models providing different patterns of independence can be employed. Classification as used herein also is inclusive of statistical regression that is utilized to develop models of priority.

As will be readily appreciated from the subject specification, the subject innovation can employ classifiers that are explicitly trained (e.g., via a generic training data) as well as implicitly trained (e.g., via observing user behavior, receiving extrinsic information) so that the classifier is used to automatically determine according to a predetermined criteria which answer to return to a question. For example, with respect to SVM's that are well understood, SVM's are configured via a learning or training phase within a classifier constructor and feature selection module. A classifier is a function that maps an input attribute vector, x=(x1, x2, x3, x4, xn), to a confidence that the input belongs to a class—that is, f(x)=confidence(class).

As used herein, the term “inference” refers generally to the process of reasoning about or inferring states of the system, environment, and/or user from a set of observations as captured via events and/or data. Inference can be employed to identify a specific context or action, or can generate a probability distribution over states, for example. The inference can be probabilistic—that is, the computation of a probability distribution over states of interest based on a consideration of data and events. Inference can also refer to techniques employed for composing higher-level events from a set of events and/or data. Such inference results in the construction of new events or actions from a set of observed events and/or stored event data, whether or not the events are correlated in close temporal proximity, and whether the events and data come from one or several event and data sources.

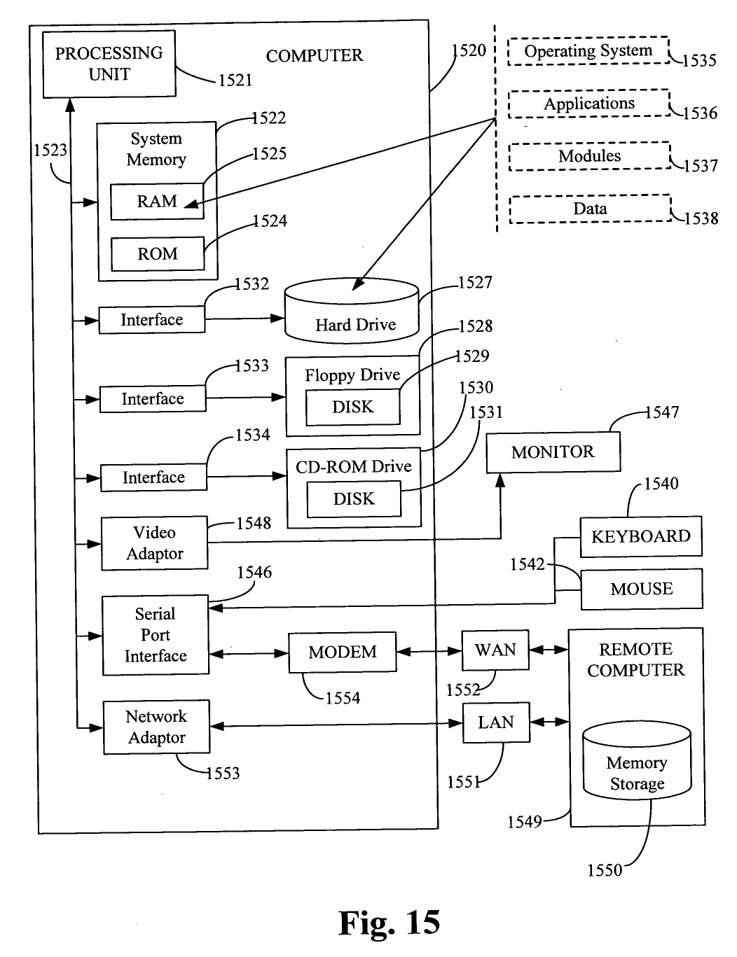

Referring now to FIG. 15, a brief, general description of a suitable computing environment is illustrated wherein the various aspects of the subject innovation can be implemented. While some aspects of the innovation has been described above in the general context of computer-executable instructions of a computer program that runs on a computing unit and/or computers, those skilled in the art will recognize that the innovation can also be implemented in combination with other program modules. Generally, program modules include routines, programs, components, data structures, etc. that perform particular tasks and/or implement particular abstract data types. Moreover, those skilled in the art will appreciate that the inventive methods can be practiced with other computer system configurations, including single-processor or multiprocessor computer systems, minicomputers, mainframe computers, as well as personal computers, hand-held computing devices, microprocessor-based or programmable consumer electronics, and the like. As explained earlier, the illustrated aspects of the innovation can also be practiced in distributed computing environments where tasks are performed by remote processing devices that are linked through a communications network. However, some, if not all aspects of the innovation can be practiced on stand-alone computing units. In a distributed computing environment, program modules can be located in both local and remote memory storage devices. The exemplary environment includes a computing unit 1520, including a processing unit 1521, a system memory 1522, and a system bus 1523 that couples various system components including the system memory to the processing unit 1521. The processing unit 1521 can be any of various commercially available processors. Dual microprocessors and other multi-processor architectures also can be used as the processing unit 1521.

The system bus can be any of several types of bus structure including a USB, 1394, a peripheral bus, and a local bus using any of a variety of commercially available bus architectures. The system memory may include read only memory (ROM) 1524 and random access memory (RAM) 1525. A basic input/output system (BIOS), containing the basic routines that help to transfer information between elements within the computing unit 1520, such as during start-up, is stored in ROM 1524.

The computing unit 1520 further includes a hard disk drive 1527, a magnetic disk drive 1528, e.g., to read from or write to a removable disk 1529, and an optical disk drive 1530, e.g., for reading from or writing to a CD-ROM disk 1531 or to read from or write to other optical media. The hard disk drive 1527, magnetic disk drive 1528, and optical disk drive 1530 are connected to the system bus 1523 by a hard disk drive interface 1532, a magnetic disk drive interface 1533, and an optical drive interface 1534, respectively. The drives and their associated computer-readable media provide nonvolatile storage of data, data structures, computer-executable instructions, etc. for the computing unit 1520. Although the description of computer-readable media above refers to a hard disk, a removable magnetic disk and a CD, it should be appreciated by those skilled in the art that other types of media which are readable by a computer, such as magnetic cassettes, flash memory cards, digital video disks, Bernoulli cartridges, and the like, can also be used in the exemplary operating environment, and further that any such media may contain computer-executable instructions for performing the methods of the subject innovation. A number of program modules can be stored in the drives and RAM 1525, including an operating system 1535, one or more application programs 1536, other program modules 1537, and program data 1538. The operating system 1535 in the illustrated computing unit can be substantially any commercially available operating system.

A user can enter commands and information into the computing unit 1520 through a keyboard 1540 and a pointing device, such as a mouse 1542. Other input devices (not shown) can include a microphone, a joystick, a game pad, a satellite dish, a scanner, or the like. These and other input devices are often connected to the processing unit 1521 through a serial port interface 1546 that is coupled to the system bus, but may be connected by other interfaces, such as a parallel port, a game port or a universal serial bus (USB). A monitor 1547 or other type of display device is also connected to the system bus 1523 via an interface, such as a video adapter 1548. In addition to the monitor, computers typically include other peripheral output devices (not shown), such as speakers and printers.

The computing unit 1520 can operate in a networked environment using logical connections to one or more remote computers, such as a remote computing unit 1549. The remote computing unit 1549 may be a workstation, a server computer, a router, a peer device or other common network node, and typically includes many or all of the elements described relative to the computing unit 1520, although only a memory storage device 1550 is illustrated in FIG. 15. The logical connections depicted in FIG. 15 may include a local area network (LAN) 1551 and a wide area network (WAN) 1552. Such networking environments are commonplace in offices, enterprise-wide computer networks, Intranets and the Internet.

When employed in a LAN networking environment, the computing unit 1520 can be connected to the local network 1551 through a network interface or adapter 1553. When utilized in a WAN networking environment, the computing unit 1520 generally can include a modem 1554, and/or is connected to a communications server on the LAN, and/or has other means for establishing communications over the wide area network 1552, such as the Internet. The modem 1554, which can be internal or external, can be connected to the system bus 1523 via the serial port interface 1546. In a networked environment, program modules depicted relative to the computing unit 1520, or portions thereof, can be stored in the remote memory storage device. It will be appreciated that the network connections shown are exemplary and other means of establishing a communications link between the computing units can be employed.

In accordance with the practices of persons skilled in the art of computer programming, the subject innovation has been described with reference to acts and symbolic representations of operations that are performed by a computer, such as the computing unit 1520, unless otherwise indicated. Such acts and operations are sometimes referred to as being computer-executed. It will be appreciated that the acts and symbolically represented operations include the manipulation by the processing unit 1521 of electrical signals representing data bits which causes a resulting transformation or reduction of the electrical signal representation, and the maintenance of data bits at memory locations in the memory system (including the system memory 1522, hard drive 1527, floppy disks 1529, and CD-ROM 1531) to thereby reconfigure or otherwise alter the computing unit system's operation, as well as other processing of signals. The memory locations wherein such data bits are maintained are physical locations that have particular electrical, magnetic, or optical properties corresponding to the data bits.

Although the innovation has been shown and described with respect to certain illustrated aspects, it will be appreciated that equivalent alterations and modifications will occur to others skilled in the art upon the reading and understanding of this specification and the annexed drawings. In particular regard to the various functions performed by the above described components (assemblies, devices, circuits, systems, etc.), the terms (including a reference to a “means”) used to describe such components are intended to correspond, unless otherwise indicated, to any component which performs the specified function of the described component (e.g., that is functionally equivalent), even though not structurally equivalent to the disclosed structure, which performs the function in the herein illustrated exemplary aspects of the innovation. Furthermore, to the extent that the terms “includes”, “including”, “has”, “having”, and variants thereof are used in either the detailed description or the claims, these terms are intended to be inclusive in a manner similar to the term “comprising.”

What is claimed is:

1. A system for an ambulatory patient treatment, comprising:
a modular component(s) as part of a distributed computing arrangement, the modular component including a plurality of cards replaceable based on real time biometric data monitoring requirements of the ambulatory patient; and
a control system that regulates the modular component(s) for at least one of biometric data acquisition and drug delivery to the ambulatory patient.

2. The system of claim 1, the modular component further comprising a common data controller that interacts with a modality specific circuitry for collection of biometric data.

3. The system of claim 2, the control system is a Master Controller and the common data controller includes a bus interface that coordinates transmittal of biometric data to the Master Controller.

4. The system of claim 2, the common data controller includes a clock that supplies the modular component with a programmable data acquisition rate different than another modular component of the distributed computing arrangement.

5. The system of claim 1 further comprising an insertable communication card that configures communication between the modular component and the control system, to a predetermined protocol.

6. The system of claim 2 further comprising amplifiers with auto-ranging gain sets to facilitate biometric data acquisition during rest and exercise periods of the ambulatory patient.

7. The system of claim 1 further comprising a plurality of clients in wireless communication with the modular component for a monitor of biometric data.

8. The system of claim 1 further comprising a belt wearable by the ambulatory patient, the belt hosts the plurality of modular components.

9. The system of claim 1 further comprising an artificial intelligence component trainable for drug delivery and biometric data acquisition.

10. A method of biometric data acquisition, comprising:
acquiring a first biometric indicia via a first modality specific module of a modular component that forms a distributed computing network around a patient;
replacing the first modality specific module with a second modality specific module, the second modality specific module measures a second biometric indicia, and
transmitting the first biometric indicia and second biometric indicia for a real time monitoring thereof.

11. The method of claim 10 further comprising administering drug delivery to a patient via I/O controls of the modular component.

12. The method of claim 10 further comprising acquiring data by the modular component at a rate different than another modular component associated with the distributed computing network.

13. The method of claim 10 further comprising auto-ranging an amplifier with adjustable gain sets to accommodate for level of patient's activity.

14. The method of claim 10 further comprising configuring communication between the modular component and the control system to a predetermined protocol.

15. The method of claim 10 further comprising employing programmable filters for real time data filtering.

16. The method of claim 10 further comprising real time biometric data streaming to clients.

17. The method of claim 10 further comprising adjusting rate of data acquisition based on feed back from users.

18. The method of claim 10 further comprising controlling modular components via a maser controller operatively connected thereto.

19. The method of claim 10 further comprising employing artificial intelligence components to facilitate data acquisition and drug delivery.

20. A system for an ambulatory patient treatment, comprising:
collecting means for acquiring biometric data from an ambulatory patient; and
means for scaling the collecting means.
